(19) United States
(12) Patent Application Publication
(10) Pub. No.: US 2011/0165662 A1
(43) Pub. Date: Jul. 7, 2011

(54) METHOD FOR HARVESTING MICROALGAE SUSPENDED IN AN AQUEOUS SOLUTION USING A HYDROPHOBIC CHEMICAL

(75) Inventors: William W. Berry, Lakeland, FL (US); Mark G. Tegen, Gig Harbor, WA (US); William Rusty Sutterlin, Hoover, AL (US)

(73) Assignee: Inventure Chemical, Inc., Gig Harbor, WA (US)

(21) Appl. No.: 12/835,327

(22) Filed: Jul. 13, 2010

Related U.S. Application Data
(60) Provisional application No. 61/225,097, filed on Jul. 13, 2009.

(51) Int. Cl. C12N 1/12

(52) U.S. Cl. 435/257.1

(57) ABSTRACT

A process for harvesting microalgae from an aqueous suspension of microalgae is disclosed. A dilute aqueous suspension of the algae is mixed with a hydrophobic liquid with a specific gravity of less than 1 and, optionally, a flocculent. When the mixture settles, the microalgae become suspended in the hydrophobic liquid above the aqueous solution. The hydrophobic liquid can skimmed from the aqueous solution for further processing.

### CROSS-REFERENCE TO RELATED APPLICATIONS

This application claims priority to U.S. Provisional Application No. 61/225,097 filed Jul. 13, 2009 which is incorporated herein in its entirety.

### FIELD OF THE INVENTION

The invention relates to methods for the harvesting of microalgae from a dilute aqueous solution of microalgae using a hydrophobic chemical.

### BACKGROUND OF THE INVENTION

Commercially, microalgae are normally grown in specially constructed open outdoor ponds or closed pond photo-bioreactors (PBRs). Productivity from open ponds and closed PBRs can vary substantially. However, the benefits of using salt water, non farm land and waste CO₂ to grow vast amounts of potentially lipid and carbohydrate rich biomass are tremendous. Algae grown for biofuel production have the potential to yield from 100,000 pounds up to 500,000 pounds or more of non food biomass per acre per year.

Outdoor open ponds for intensive aquaculture typically are somewhat expensive and are frequently constructed of concrete and lined with plastic. Brine depth generally is controlled at 20 centimeters, which has been considered to be the optimum depth for producing algal biomass. A number of configurations of the ponds have been proposed for intensive aquaculture.

Open air raceway ponds are typically the most important commercially. Raceway ponds employ paddle wheels to provide mixing. Chemical and biological parameters are carefully controlled, including salt and fertilizer concentrations, pH of the brine, and purity of the culture.

Closed photo-bioreactor systems can vary widely in design and operation but all operate on the principle of establishing a controlled environment where the select algae can be maintained with prolonged contact with CO₂ without the threat of species invasion or contamination.

Microalgae is typically harvested when its concentration is in the range of <1 wt % to 4 wt % solids, since a concentration of greater than roughly 4 wt % of microalgae will typically start to impede growth and kill the microalgae. Such dilute cultures of microalgae are generally uneconomical to process in part because separating the algae from the brine in which they grow is difficult. The algae have mobility, neutral density, and a small elliptical shape of approximately <1 to 16 microns that make the algae somewhat difficult to harvest. In addition, the volume of brine to microalgae is roughly 10:1, so the process of water removal can amount to a substantial cost in particular when using clarifiers, centrifuges, hydrocyclones, filter presses, drum dryers and/or spin flash dryers. For example in the case of a 10:1 ratio, if the volume of algae to be harvested is 500,000 lbs, then 5,000,000 lbs of water would need to be processed to gather the algae.

Thus, there is a need for a more economical and efficient method for harvesting microalgae without the use of excessive energy for drying or the cost associated with operating physical equipment to remove the microalgae from a solution containing dilute microalgae and brine.

### SUMMARY OF THE INVENTION

The present invention includes a process of separating microalgae suspended in an aqueous solution from the aqueous solution comprising (a) mixing the aqueous suspension with a hydrophobic liquid and, optionally, a flocculent and (b) incubating the mixture to form a top phase containing the hydrophobic liquid and at least a portion of the algae and a bottom phase containing the aqueous solution. The aqueous suspension can be a growth medium for the algae and contain up to or equal to 4 wt % microalgae. The hydrophobic liquid can be, for example, fatty acid methyl ester (FAME) present in a weight ratio of hydrophobic liquid to the microalgae from 1:1 to 100:1. The top phase can skimmed from the bottom phase and the algae in the top phase can be separated from the hydrophobic liquid. Both the hydrophobic liquid and the bottom aqueous phase can be reused for further separations and as growth medium, respectively.

### BRIEF DESCRIPTION OF THE DRAWINGS

FIG. 1 is a process schematic demonstrating the flow of algae, water/brine, and hydrophobic fluid during the separation process.

FIG. 2 is a photograph showing 3 types of algae (from left to right: Chlorella, Nannochloropsis, and cyanobacteria Spirulina) concentrated in the top phase of FAME above a cloudy emulsion containing water and FAME.

### DETAILED DESCRIPTION

The invention provides a process for separating microalgae from the medium in which they grow by mixing the medium with a hydrophobic liquid and allowing the resulting mixture to settle creating a top phase, containing the hydrophobic liquid and microalgae, and a bottom phase, containing the aqueous growth medium. The resulting mixture of microalgae in the hydrophobic liquid can be concentrated from 1% solids in aqueous solution to 10% solids in the hydrophobic liquid and the volume of liquid can be reduce 10-fold. Thus, the invention is capable of economically dewatering algae obtained from open ponds or PBRs by reducing the volume of fluid to process and by removing the overwhelming majority of the brine water from the algae in one simple low energy mixing step. The recovered algae, which can be in the presence of both residual FAME and intracellular water, can be processed to yield valuable commodities such as fatty acid alkyl esters for use as biodiesel as disclosed in U.S. Publication Nos. 2008/0241902 and 2009/0198077.

A suspension of microalgae in aqueous solution for separation can be obtained from any source. For example, algae suspended in their aqueous growth medium can be obtained from an open pond, PBR, or a naturally occurring ocean or lake, such as the brines of the Great Salt Lake in Utah. The microalgae can be a mixture of several different algae or a single type of algae. Examples of types of microalgae that can be used in this process include fresh water algae, salt water algae, prokaryotic algae, or a combination thereof. Cyanobacteria, Chlorophyta, diatoms, Chlorella, Nannochloropsis, cyanobacteria Spirulina, Skeletonema, and a combination thereof are microalgae that can be used in this process. In particular, cyanobacteria, Chlorophyta, and diatoms are microalgae that can be used in the process. The aqueous solution, such as growth medium, can be seawater or fresh water and can contain from about 0 wt % to about 4 wt % dissolved salts. Seawater can contain from about 3.1 wt % to about 3.8 wt % dissolved salts. The majority of the dissolved salts can be sodium chloride. Additional ions in the aqueous solution include magnesium, sulfate, calcium, inorganic carbon, potassium, boron, bromine, strontium, and fluoride. The concentration of algae in the aqueous solution is less than or equal to 4 wt %, less than or equal to 3 wt %, less than or equal to 2 wt %, or less than or equal to 1 wt % of the aqueous solution.

Separation process initiates when the microalgae suspension is mixed with a hydrophobic liquid. The hydrophobic liquid can have a specific gravity of less than 1. Examples of a hydrophobic liquid include a fatty acid alkyl ester, such as fatty acid methyl ester (FAME) or fatty acid ethyl ester, a free fatty acid, a mixture of free fatty acids, a monoglyceride, a diglyceride, a triglyceride, an alkane, or a combination thereof. The hydrophobic liquid is mixed with the microalgae suspension at from about a 1:1 to about a 100:1 weight ratio of hydrophobic liquid to algae solids; or from about a 1:1 to about a 10:1 weight ratio of hydrophobic liquid to algae solids.

A flocculent can also be added to the suspension to facilitate the separation of the algae from the aqueous solution. The flocculent can be a multivalent cation. In some cases, the flocculent can be aluminum ions, iron ions, calcium ions, magnesium ions, alum, aluminum chlorohydrate, aluminum sulfate, calcium oxide, iron(III) chloride, iron(II) sulfate, polyacrylamide, latex, sodium aluminate, sodium silicate, and a combination thereof. The flocculent can change the density, the polarity, or both of the suspension. A flocculent can be added to the suspension in an amount from about 0.1 to about 10 wt %, from about 0.1 to about 5 wt %, from about 0.5 to about 1.5 wt %, or of about 1 wt % of the suspension. Flocculent can also be added in an amount from about a 1:1 to about a 100:1 weight ratio of flocculent to algae solids; or from about a 1:1 to about a 10:1 weight ratio of hydrophobic liquid to algae solids.

A component can also be added to change the pH of the solution to facilitate separation of algae from the aqueous solution. For example, a base, such as sodium bicarbonate, (ammonium carbonate) or ammonium nitrate, can be added to change the pH of the solution to from about pH 7 to about pH 8.

The resulting mixture containing the aqueous microalgae suspension, the hydrophobic liquid, and, optionally, the flocculent is mixed. The mixture can be vigorously mixed to homogeneity. The mixture is then incubated for at least about 30 seconds to about 12 hours, at least about 30 seconds to about 1 hour, at least about 30 seconds to about 10 minutes at or about at room temperature, such as 15° C. After incubation, the mixture separates into a top and bottom phase. The bottom phase contains an aqueous solution that sinks due to gravity and polarity and the top phase contains the algae and the hydrophobic liquid. The algae can be present in the top phase in an amount of at least 50 wt %, 55 wt % , 60 wt %, 65 wt %, 70 wt %, 75 wt %, 80 wt %, 85 wt %, 90 wt %, 95 wt %, or 100 wt % of the total algae in the mixture.

The top phase can be removed by skimming using a common oil/water separation device such as an API oil/water separator (available from Hydrasep, Hernando, MS) or other skimming device. The bottom phase containing the aqueous solution can be reused as a growth medium. The skimmed top phase can be transferred to a concentrating device such as a centrifuge or filter press to separate the hydrophobic liquid from the microalgae. The resulting microalgae are now usable for further processing and the hydrophobic liquid can be reused to separate additional algae. The microalgae from the skimmed top phase can contain both residual hydrophobic liquid (such as FAME) and water and can be further processed such as disclosed in U.S. Publication Nos. 2008/0241902 and 2009/0198077.

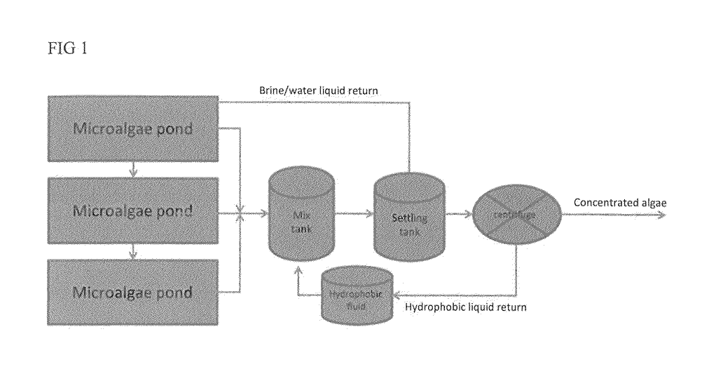

The steps of a generalized process in accordance with the invention for separating algae from the medium in which they are growing are represented in FIG. 1. A dilute stream of microalgae in growth medium and a hydrophobic liquid are channeled to a mixing tank to allow for the continuous harvesting of the microalgae solids. The mixture is then transferred to a settling tank. Alternatively, the mixture can settle in the mixing tank. After settling, the top phase is skimmed and centrifuged to concentrate the algae. The hydrophobic liquid is returned to the mixing tank for reuse and the aqueous bottom phase is returned to the microalgae pond for reuse.

It is to be understood that the scope of the present invention is not to be limited to the specific embodiments described. The invention may be practiced other than as particularly described and still be within the scope of the accompanying claims.

All referenced cited herein, including all patents, published patent applications, and published scientific articles, are incorporated by reference in their entireties for all purposes.

### EXAMPLES

### Example 1

### Separation of 1 wt % to 4 wt % algae from a Brine Solution using 1 wt % to 4 wt % FAME

FAME was added to a solution of seawater (approximately 3.5 wt % dissolved salt) and Chlorella algae in the amounts shown in Table 1. The solution was mixed using an electric mixer for 5 minutes at 20° C. and was allowed to separate for 5 minutes. The amount of algae in the brine and in the FAME layers was determined by drying a volumetric sample of the brine prior to FAME addition and after separation and determining its algae content.

As shown in Table 1, a 1:1 ratio by weight of algae to FAME in a solution of brine results in the algae separating into the FAME layer.

TABLE 1
Separation of 1 wt % to 4 wt % algae from a brine solution using 1 wt %
to 4 wt % FAME

| | Test 1 | Test 2 | Test 3 | Test 4 |
|---|---|---|---|---|
| Algae (solids | 1 | 2 | 3 | 4 |
| %/wt) | | | | |
| Brine (%/wt) | 98 | 96 | 94 | 92 |
| FAME %/wt | 1 | 2 | 3 | 4 |
| Results | | | | |
| wt % algae in | ND | ND | ND | ND |
| brine | | | | |
| wt % algae in | 50 | 50 | 50 | 50 |
| FAME | | | | |

ND = not detected

### Example 2

### Use of FAME to Separate of Different Types of Algae from Water

Three different types of algae were used to demonstrate the use of FAME to separate algae from water: Chlorella, Nannochloropsis, and cyanobacteria Spirulina. For each strain, 15 grams of dry algae and 400 grams of distilled water were mixed using a high-sheer mixer to reestablish a “pond-like” setting such that a colloid of algae in water was formed. The cyanobacteria Spirulina was of a larger particle size than the other two strains of algae. During harvesting, the particles most likely clumped together. Thus, to reestablish the original setting, it was necessary to return the particles to their original size. Thus, prior to the experiment, the algae were sieved and particles ranging from 53 to 125 μm in diameter were used in the experiment rather than the bulk sample.

The colloids were allowed to settle for approximately 12 hours. A thin layer of FAME (approximately 60 grams) was then added to the algae in water solution such that the solution was approximately 84 wt % water, 3 wt % algae and 13 wt % FAME. The solution was then mixed at 9500 revolutions per minute with a high-sheer mixer to form an emulsion. The solution was then allowed to separate for 12 hours.

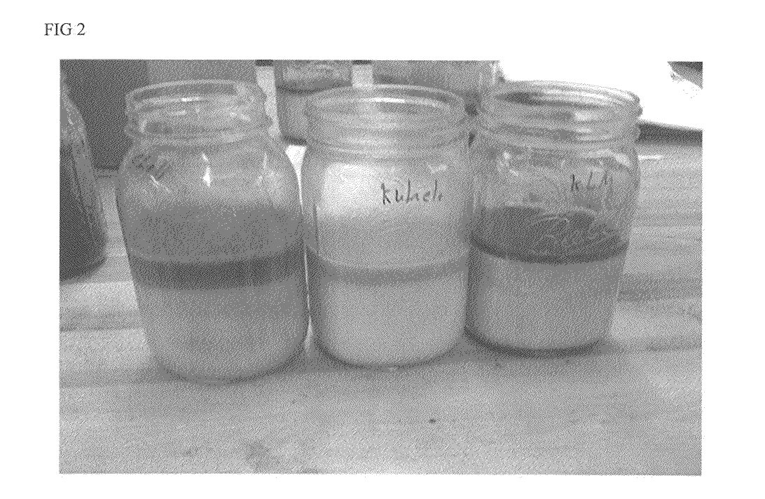

FIG. 2 is a photograph, taken after separation, showing the 3 algae (from left to right: Chlorella, Nannochloropsis, and cyanobacteria Spirulina) concentrated in the top phase of FAME above a cloudy emulsion containing water and FAME.

The FAME layer was skimmed and the FAME was separated from the algae. The algae were then weighed to determine the amount of algae in the FAME and aqueous layers. 100% of the Chlorella sample was collected in the FAME layer. 85-90% of the Nannochloropsis sample was collected in the FAME layer. 65-70% of the cyanobacteria Spirulina sample was collected in the FAME layer.

What is claimed is:

1. A process for separating microalgae suspended in an aqueous solution from the aqueous solution comprising:
(a) mixing the microalgae suspended in the aqueous solution with a hydrophobic liquid and, optionally, a flocculent; and
(b) incubating the mixture of step (a) to form a top phase comprising the hydrophobic liquid and at least a portion of the microalgae and a bottom phase comprising the aqueous solution.

2. The process of claim 1, wherein the algae is capable of growing in the aqueous solution.

3. The process of claim 1, wherein the weight percentage of the microalgae in the aqueous solution prior to mixing with the hydrophobic liquid is from less than or equal to 4 wt %.

4. The process of claim 1, wherein the weight ratio of the hydrophobic liquid to the microalgae in step (a) is in from 1:1 to 100:1.

5. The process of claim 1, wherein the hydrophobic liquid has a specific gravity of less than 1.0.

6. The process of claim 1, wherein the hydrophobic liquid is a fatty acid alkyl ester, a free fatty acid, a mixture of free fatty acids, a monoglyceride, a diglyceride, a triglyceride, an alkane, or a combination thereof.

7. The process of claim 1, wherein the hydrophobic liquid is a fatty acid methyl ester (FAME).

8. The process of claim 1, wherein the aqueous suspension is mixed with the hydrophobic liquid and the flocculent.

9. The process of claim 8, wherein the flocculent comprises a multivalent cation.

10. The process of claim 8, wherein the flocculent comprises aluminum ions, iron ions, calcium ions, magnesium ions, alum, aluminum chlorohydrate, aluminum sulfate, calcium oxide, iron(III) chloride, iron(II) sulfate, polyacrylamide, latex, sodium aluminate, sodium silicate, or a combination thereof.

11. The process of claim 8, wherein the flocculent is in an amount from a 1:1 to a 100:1 weight ratio of the flocculent to the microalgae in step (a).

12. The process of claim 1, further comprising (c) skimming the top phase from the bottom phase.

13. The process of claim 12, further comprising (d) centrifuging the top phase to separate the hydrophobic liquid from the algae.

14. The process of claim 13, further comprising (e) reusing the hydrophobic liquid to separate algae suspended in a second aqueous solution.

15. The process of claim 14, further comprising (f) reusing the bottom phase for growing additional algae.

16. The process of claim 1, wherein the portion of the microalgae in the top phase is at least 50 wt % of the microalgae suspended in the aqueous solution in step (a).

17. The process of claim 1, wherein the aqueous solution of step (a) contains from 0 wt % to 4 wt % dissolved salts.
